(19) United States
(12) Patent Application Publication
(10) Pub. No.: US 2009/0076597 A1
(43) Pub. Date: Mar. 19, 2009

(54) System for mechanical adjustment of medical implants

(76) Inventors: Jonathan Micheal Dahlgren, Surrey (CA); Daniel Gelbart, Vancouver (CA)

Correspondence Address: DAN GELBART, 4706 DRUMMOND DR., VANCOUVER, BC, V6T-1B4, CA

(21) Appl. No.: 11/902,099

(22) Filed: Sep. 19, 2007

(51) Int. Cl. A61F 2/24

(52) U.S. Cl. 623/2.1; 600/12; 606/53

(57) ABSTRACT

A system for mechanically adjusting medical implants uses an external coil to set up a magnetic field. The magnetic field causes an actuator inside the implant to move in small steps, allowing fine adjustment. The element responding to the magnetic field can be magnetostrictive or SMA based. Large motions are made up from small steps by using two one-way clutches allowing the active element to move small increments in one direction. For SMA based devices, short burst of AC magnetic field are used. For magnetostrictive devices short pulse of unipolar magnetic field are used.

### FIELD OF THE INVENTION

The invention is in the medical field and in particular in the area of implants requiring adjustment after implantation.

### BACKGROUND OF THE INVENTION

Many implanted medical devices can benefit from ability to be adjusted after implantation, particularly if the adjustment can be done externally without the need of surgery. For example, when a cardiac valve is failing sometimes an adjustment ring or device is installed in order to restore the failing valve to the correct shape. The well known example is the annuloplasty ring used for mitral valve repair. Such rings are normally installed by using open heart surgery, but percutaneous techniques have been developed recently. It is desirable to be able to adjust such a ring in the future without further invasive procedures, since the condition of the valve may deteriorate. For example, valve annulus may dilate further causing incomplete closure of the two valve leaflets.

Another example is spine and bone curvature correction devices in orthopedic surgery, which have to be periodically adjusted in order to allow the body to gradually accommodate to the changes. Still another example is gastric restrictors which can benefit from later date adjustment. Some prior art Shape Memory Alloy (SMA) actuators can be heated by electrical induction heating from the outside of the body. They use the type of SMA wire that has a non-reversible transformation when heated and stays in the new shape after cooling down. SMA belongs to the family of Nitinol alloys that is well known in medicine and is used for self-expanding stents. Remotely controlled SMA actuators have two major disadvantages. First, they can not be controlled well, as a few degrees difference in heating can make the difference from no motion to full deformation. Secondly, in order to respond to induction heating or any electromagnetic coupling a closed path is required for the current to flow. The SMA part acts as a short circuited secondary coil of a transformer. Such a closed path causes major problems when the patient has to undergo a Magnetic Resonance Imaging (MRI) scan. The MRI machine uses a combination of a static magnetic field and a pulsating high powered RF field. The RF field induces a secondary current in any conductive object with a closed electrical path. It is desired to have a remotely adjustable implant capable of accurate mechanical adjustment while maintaining compatibility with MRI systems.

It is also desirable to be able to make the mechanical adjustment by a large number of small equal steps. In some applications a bi-directional adjustment is desirable. The following disclosure describes a system that among other features addresses these problems.

### SUMMARY OF THE DISCLOSURE

A system for mechanically adjusting medical implants uses an external coil to set up a magnetic field. The magnetic field causes an actuator inside the implant to move in small steps, allowing fine adjustment. The element responding to the magnetic field can be magnetostrictive or SMA based. Large motions are made up from small steps by using two one-way clutches allowing the active element to move small increments in one direction. For SMA based devices, short burst of AC magnetic field are used. For magnetostrictive devices short pulse of unipolar magnetic field are used.

### BRIEF DESCRIPTION OF THE DRAWINGS

FIG. 1A is a longitudinal section of the stepping actuator using SMA wire.

FIG. 1B is a longitudinal section of the stepping actuator using Terfenol.

FIG. 2A is a view of a one-way clutch using elastic elements.

FIG. 2B is a view of a one-way clutch using spring loaded wedges.

FIG. 2C is a view of a one-way clutch using spring loaded balls.

FIG. 3 is a top view of a mitral valve being repaired using the disclosed system.

FIG. 4 depicts a percutaneous delivery of a mitral valve repair system.

FIG. 5 is a longitudinal section of a bi-directional adjustment system.

FIG. 6 is a graph showing the relationship between magnetic field and strain in Terfenol-D.

FIG. 7 is a longitudinal section of the stepping actuator used to adjust bone spacing.

FIG. 8 is a side view of a spine showing the disclosed system remotely adjusted to correct the curvature of the spine.

FIG. 9 is a longitudinal section of a bi-directional actuator.

### DETAILED DESCRIPTION

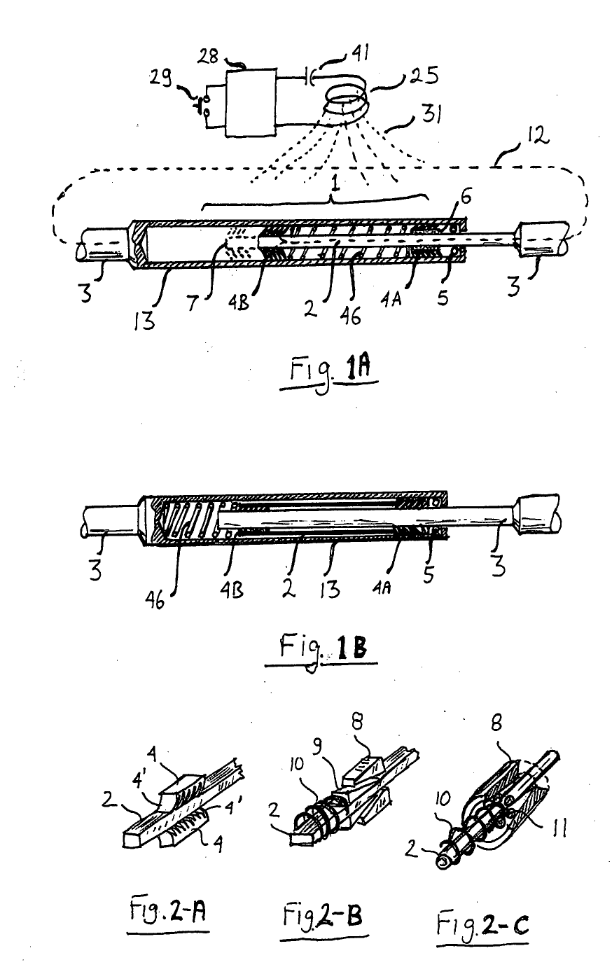

Referring to FIGS. 1A and 1B, a stepping actuator 1 contains element 2 capable of changing length as a response to changes in an external magnetic field or in response to heating induced by a changing magnetic field. Element 2 can be made of a highly magnetostrictive alloy such as Terfenol-D or from a Shape Memory Alloy (SMA) such as specially treated Nitinol. Terfenol-D is commercially available in a wide range of sizes from Etrema (www.etrema-usa.com). It can change length by up to 0.15% in response to a magnetic field of about 0.3 Tesla. Depending on the crystal orientation it can be made to increase or decrease length when magnetized. Newer types of magnetostrictive alloys, such as Ni—Mn—Ga alloy can be used for larger motions than Terfenol-D but they are not as readily available. SMA actuator wires, also known as “muscle wires”, “Nitinol actuator wire” and “Flexinol”, contract by up to 5% when heated and return to the original length when allowed to cool. For this disclosure the term SMA primarily refers to materials that can be cycled repeatedly by low temperature heating, not the SMA type that required “resetting” at a high temperature once heated. Actuation can be done remotely by using an AC magnetic field to induce a current heating the SMA wire, similar to an air-core transformer with a shorted secondary winding. When heated, the SMA wire shortens by about 5%. SMA actuator wire is readily available in a wide range of sizes from Dynalloy and other suppliers (www.dynalloy.com). In order to achieve an accurate and repeatable adjustment, actuator 1 moves in small steps while holding its position during and in between steps. Referring now to FIG. 1A showing an SMA version of actuator, SMA wire 2 connected to implant 3 is entering tube 13. Two one way clutches, 4A and 4B attached to the wire 2 allow the wire to move only in one direction, into the tube. When the section of wire 2 between clutches 4A and 4B is repeatedly expanding and contracting, wire 2 will move in one direction to a new position 7. Compression spring 46 keeps wire 2 under tension. The principle of converting small back and forth motion into a large unidirectional motion is well known in mechanical engineering. A seal 5, typically made of Teflon or silicone rubber, can be used to prevent tissue cells or blood cells from entering tube 13. Pure liquid, such as blood plasma or saline solution inside actuator will not affect operation significantly; therefore seal does not have to be truly hermetic. When an SMA based actuator is used, a closed electrical path 12 has to exist connecting implant parts 3. To assure the induced current will flow through wire 2, clutch 4A needs to be attached but electrically insulated from wire 2 by insulating sleeve 6 or any other means. The induced current travels via loop 12, implant 3, tube 13, clutch 4B and wire 2, returning to implant 3. To increase the coupling efficiency between the external coil 25 and actuator 1, coil 25 can be resonated with capacitor 41 when connected to power source 28. When switch 29 is closed a burst of alternating (AC) magnetic field 31 causes wire 2 to heat up. Typical temperature required is about 60 degrees C. By using repeated bursts wire 2 is moved into tube 13 in small steps. For a distance of 10 mm between clutches 4, each step is in the range of 0.1-0.5 mm. A suitable AC frequency to use is 100 KHz to 2 MHz and a burst length of 0.5-5 seconds. Coil 25 is typically 20 cm diameter and has 25-100 turns with air spaces between turns to achieve a high-Q resonant circuit. Total power coupling efficiency is 10%-20% for Q values of about 100. Power needed by actuator depends on actuator size but is typically 1-10 W.

FIG. 1B shows a similar actuator based on magnetostriction, preferably of Terfenol-D. When Terfenol based actuators are used, pulses of unipolar (DC) magnetic field are used to cause sleeve 2 to change length by about 0.1%. Much larger changes can be achieved in Ni—Mn—Ga alloys. Since Terfenol is more brittle and less corrosion resistant than SMA alloys, sleeve 2 is fully enclosed inside tube and part of implant 3 is inside tube 13. In this drawing element 2 is a tube instead of a wire, but similar designs can be based on a wire. Element 2 in these drawings is always the element capable of changing dimensions. One of the one-way clutches 4A is attached to tube 13 and clutch 4B is attached to sleeve 2. A biasing spring 46 can be added to increase performance as Terfenol has a significantly higher compressive strength than tensile strength. While the length change is smaller than that of an SMA wire, the rate at which the wire can be cycled through the changes is much higher. The reason is that no heating and cooling is involved, the main limit is the speed in which the magnetic field is increased and decreased. Stepping rates of 1 KHz are easily achieved, compared to 1 Hz which is typical for an SMA wire. For a 10 mm distance between clutches 4A and 4B, the length change is about 10 um. The ability to use a stepping mode, getting to the end value step by step, allows precise and repeatable control. The design of the external coil 25 is different for the Terfenol actuator as no high frequencies are involved. By the way of example, coil 25 has an outside diameter of 20 cm and comprises of 1000 tightly wound turns of 1 mm diameter copper wire. It is pulsed with a current of 100 A for about 1-10 mS whenever switch 29 is closed. When switch 29 is held closed pulsing continues at rate of about 20-200 Hz (0.2 mm-2 mm/sec). Capacitor 41 is not used as the coil is not resonated. To generate the high current a capacitor inside power source 28 can be discharged into the coil. A coil of these specifications will generate about 0.3 T at a distance of 6 cm from the coil. Implant 3 and tube 13 should not be made from a ferromagnetic material.

FIGS. 2A, 2B and 2C show different ways of constructing a one way clutch. In FIG. 2A the clutch 4 is a single piece flexible part having flexible teeth 4′ pressed against wire 2 at an angle. This arrangement allows wire 2 only to move in one direction. Clutch 4 can be fabricated using EDM from hardened tool steel or series 440 stainless steel.

FIG. 2B shows an embodiment using sliding wedges 9 positioned between fixed wedges 8 and wire 2. Spring 10 keeps wedges 9 preloaded. As before, wire 2 can only move in one direction.

FIG. 2C shows an embodiment using small balls 11 and a tapered hole in part 8 to replace the prismatic wedges of FIG. 2B. As before, spring 10 provides preload. The basic actuator described above can be made in different sizes and used in many different medical applications requiring a mechanical adjustment. By the way of example, two such applications are shown: a mitral valve repair and an orthopedic application. The clutches can be designed to slide on the central member 2 or attached to the central member and slide on the external housing, as in FIG. 1A.

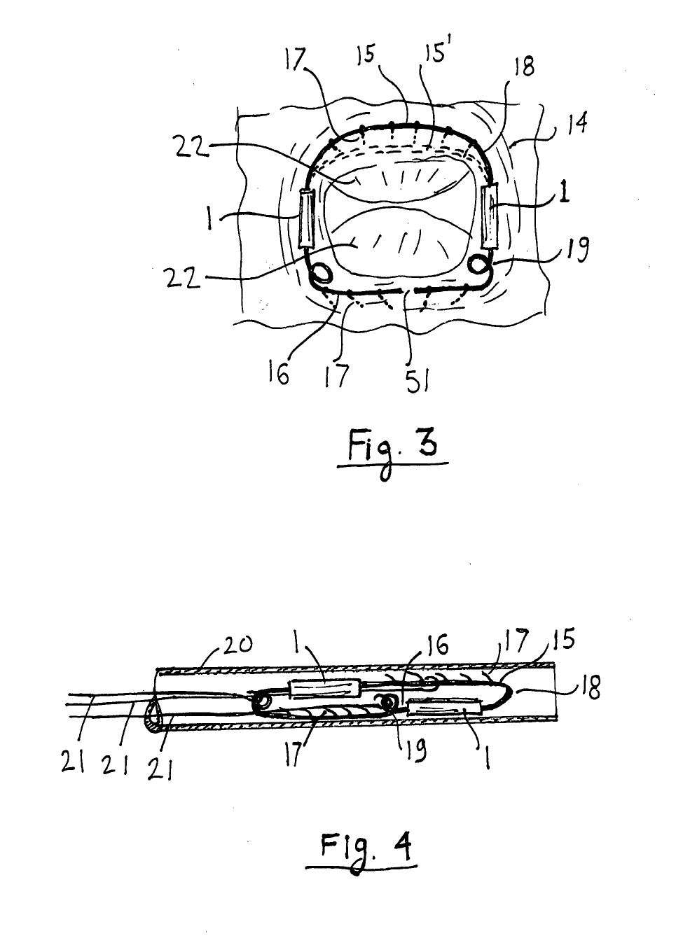

FIG. 3 shows an implant comprising of two actuators 1 and two connecting pieces 15 and 16, forming a loop around the mitral annulus 14 of a mitral valve located between the left atrium and the left ventricle of a heart. In some cases valve leaflets 22 are not sealing properly and need to be brought together, typically by fastening an angioplasty ring. This procedure requires open heart surgery. The device shown in FIG. 3 can be delivered percutaneously via a catheter and adjusted at a later date, as well as serve as an anchor for an artificial mitral valve should it be needed in future. The device is held in place by barbs 17 or an equivalent method. After deployment it can be adjusted by causing actuators 1 to pull part 15 closer to part 16, as shown by dotted line 15′. The adjustment may be done a few weeks after deployment, to allow a stronger bond to develop between the device and the mitral annulus 14. Since adjustment is done by a coil external to the body, it can be re-adjusted non-invasively at future dates. Some parts of the device are made very flexible to allow folding into a catheter. By the way of example, parts 15 and 16 can be made of Nitinol with corners made thinner as shown by 18 or adding wire loops to serve as hinge points, as shown by 19. When the actuators 1 are based on SMA it is desired to have a closed electrical loop for good coupling with the external coil. When actuators are of the magnetostrictive type it is desired to have an electrical break as shown by 51 in order to improve MRI compatibility by avoiding a loop. The break can be bridged, if desired, by a non-conductive reinforcement.

FIG. 4 shows the device folded into catheter 20. The process of catheter delivery is well known in the art of cardiology and need not be detailed here. In order to position the device, typically with the aid of fluoroscopy, wires 21 are temporarily attached to it. After device is pushed out of catheter 20 and embedded into mitral annulus, wires 21 are disengaged and retracted through catheter 20. A typical size of actuator 1 for this application is 3 mm diameter by 20 mm long. When folded as shown in FIG. 4 the device will fit trough a size 18Fr catheter or larger catheter.

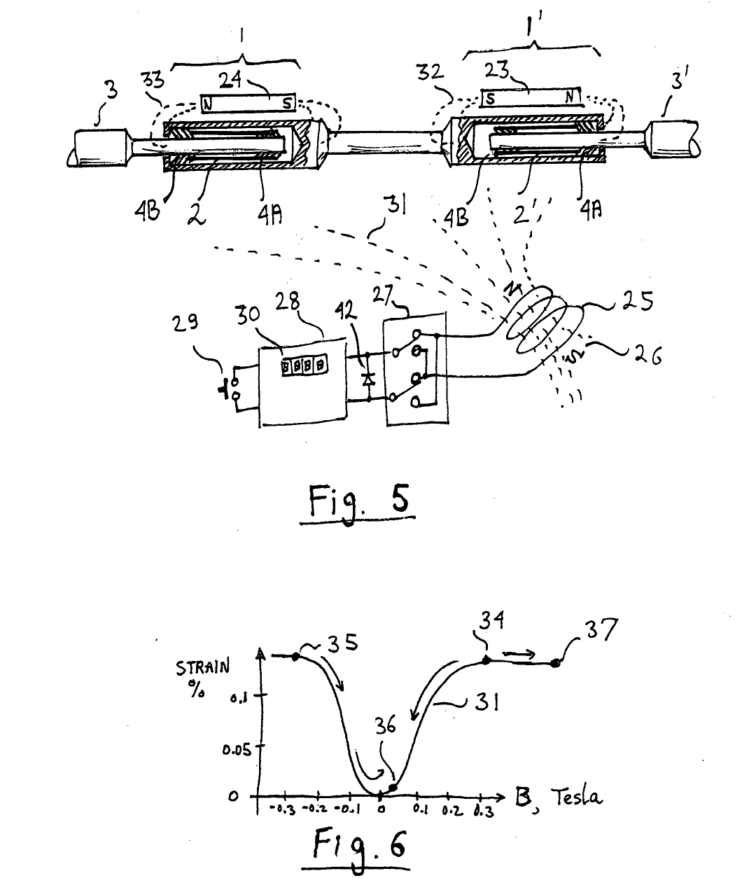

In some applications it is desired to be able to have a bi-directional remote adjustment. One method is by using two actuators operating in opposite directions. An alternative is a single actuator with bi-directional capability. FIG. 5 shows an example of bi-directional adjustment. Actuators 1 and 1′ are mounted in a manner allowing actuator 1 to pull implant 3 while actuator 1′ pushes end 3′ of same implant. As an example, if ends 3 and 3′ are the ends of a ring, activating actuator 1 will reduce the size of the ring while activating actuator 1′ will increase the size of the ring. Whether the actuator pulls or pushes is determined by the direction the one-way clutches 4A and 4B are mounted. In order to be able to activate both directions from a single coil 25, biasing magnets 23 and 24, generating magnetic fields 32 and 33, are used. When the polarity of coil 25 is as shown by 26 it will enhance the magnetization of magnet 24 and reduce the magnetization of magnet 23. When polarity is reversed by switch 27, the effect on magnets 23 and 24 is reversed. Diode 42 is used to avoid abrupt change in the current through coil 25 in order to minimize electromagnetic interference. By the way of example, closing switch 29 momentarily will send a magnetic pulse causing one of the actuators (selected by switch 27) to step a single step. Holding switch 29 closed will send a continuous pulse train for continuous stepping. Power source 28 can be equipped with display 30 showing total number of steps or total movement in any convenient units. The principle of selectively activating the desired actuator will become clear by studying FIG. 6 together with FIG. 5. FIG. 6 shows a graph of the strain (corresponding to the motion) of Terfenol-D in response to the strength of the magnetic field in units of Tesla. For either direction of magnetization the size change in the Terfenol reaches a saturation value at about 0.3 T. Magnets 23 and 24 keep Terfenol sleeves 2 and 2′ at saturation points 34 and 35 on the graph. In FIG. 5, magnetic field created by coil 25 is in the same direction as the bias magnet 24, causing the field in sleeve 2 in actuator 1 to move from point 34 on the graph to point 37. Since the Terfenol is in magnetic saturation, no mechanical movement will result. The same field causes sleeve 2′ in actuator 1′ to move from point 35 to a very low field represented by point 36. Exact cancellation of the field to zero is not important, and the zero point can be crossed by a field sufficiently strong to reverse bias sleeve 2′. This is shown by point 36. By changing the field from saturation to near zero sleeve 2′ will change dimensions and actuator 1′ will step one step. The operation is repeated until the correct position is achieved. If reverse motion is needed, polarity switch 27 is switched and actuator 1 will operate. The number of steps per second is mainly limited by the inductance and power dissipation of the coil. The same method used for bi-directional adjustments can also be used for two separate unidirectional adjustments, such as X and Y positioning, operated from a single coil. While the example is for Terfenol, similar selective activation can be used for SMA based adjustments by choosing different frequencies, different time constants etc. For example, a slow responding SMA actuator stepping 1 mm per step can be place in series with a fast responding actuator stepping 0.1 mm per step in the manner shown in FIG. 5. The response time can be adjusted by the diameter of wire 2. When short bursts of AC magnetic field are sent, the fast actuator moves in 0.1 mm steps in one direction but the slow one does not respond. When a long burst is sent, the fast actuator moves 0.1 mm and the slow actuator moves 1 mm in the opposite direction, for a total movement of 0.9 mm in the opposite direction. In order to move 0.1 mm in the direction of the slow actuator, one long burst (net movement of 0.9 mm) is followed by 8 short ones (−0.8 mm) for a total movement of 0.1 mm.

FIG. 7 shows a typical orthopedic application. An actuator 1 is wedged between two bones 47. Actuator has a wedge shaped body 48 with a pivot or flexing point 50. When rod 2 expands and contracts in response to external activation, wedge 49 is pulled into body 48 by action of one way clutch 4. An actuator as in FIG. 7 can be made from very small (a few mm) to very large (a few cm) sizes. It can be designed for percutaneous delivery by delivering it in the fully closed state and expanding it after delivery. The actuator can be based on SMA or magnetostriction, as explained earlier.

Another example is spine curvature correction shown in FIG. 8. In order to correct the shape of spine 39 an array of actuators 1 are attached to the spine by hooks 38 or any other attachment. An external coil 25 is used to periodically adjust actuators 1 in order to re-shape spine 39. A ferromagnetic core 40 is used to focus the magnetic field on the desired actuator. Core 40 is typically made of laminated silicon iron alloy similar to transformer cores. The ability to periodically adjust spine during the long reshaping period without surgery or without metal parts penetrating the skin is a major advantage. In this application a typical actuator will use a Terfenol-D core having a cross section of 1×5 mm to 3×20 mm and length of 10-50 mm. The larger cross section are used in those applications requiring considerable forces. A similar design can be based on SMA as detailed in previous examples.

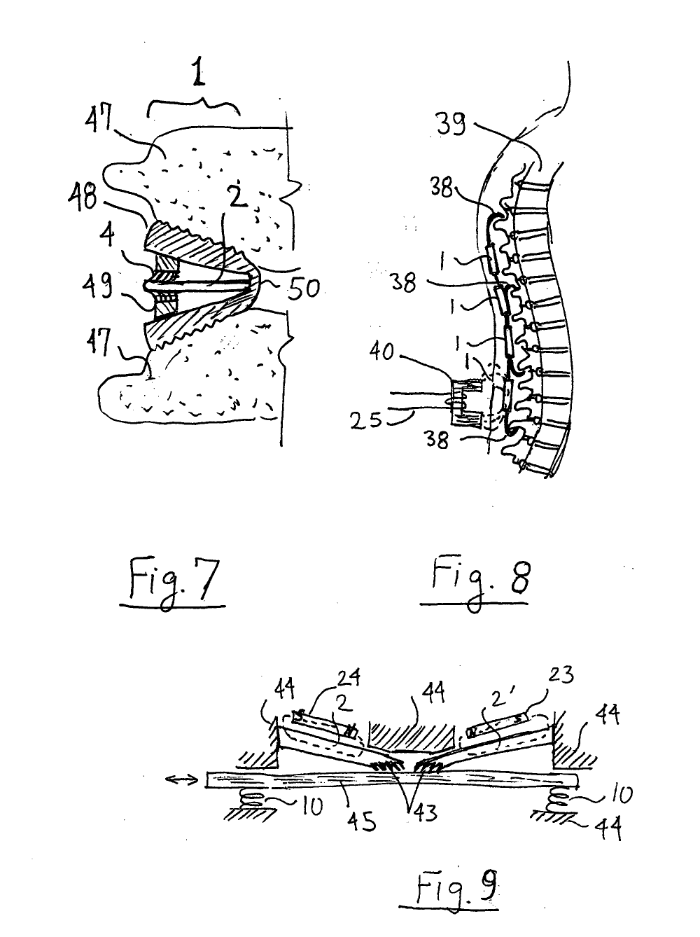

For application requiring a very large number of bi-directional adjustments, a true bi-directional design as shown in FIG. 9. Rods 2 and 2′ are made of a material capable of remotely activated dimensional change, such as SMA or Terfenol. In this figure rods 2 and 2′ are mounted to frame 44 at one end and slide against the frame at the other end. Rods 2 and 2′ elongate when activated by a magnetic field. A version based on shortening rods made of SMA clearly can be made based on the same principles. When not activated rods 2 and 2′ touch rod 45 lightly. Rod 45 is held in place by springs 10. When rod 2 or 2′ elongate they are pressed against rod 45 and move it. Teeth 43 can be added to increase friction. Magnets 23 and 24 allow operation of both direction from a single coil, as explained earlier.

An alternate embodiment replaces the Terfenol sleeve with a piezoelectric sleeve which is connected to a pick-up coil. Activating the external magnetic field induces a voltage in the pick-up coil causing the piezoelectric sleeve to change its length. The pick-up coil can be wound outside the actuator.

While all above examples describe linear motion it should be understood that they can be applied to rotary, arcuate, helical or any other kind of motion. The equivalence of rotary and linear actuators is well known in the art of actuators.

The SMA based actuators respond to the heat created by the current induced by the magnetic field. Other methods of creating heat should be considered part of the disclosure, such as ultrasonic heating or microwave heating. Some polymers have SMA-like properties and can be used as well. They allow the construction of non metallic actuators which have very good MRI compatibility. Obviously they have to be heated by methods other than inductive coupling. A narrow ultrasound beam can be used.

What is claimed is:

1. A medical implant capable of non invasive step-by-step adjustment in response to a changing external magnetic field.

2. A system for mitral valve repair including an actuator capable of step-by-step adjustment, said steps activated by a changing magnetic field.

3. A orthopedic correction system using step-by-step adjustment activated by a changing magnetic field.

4. A system as in claim 2 delivered to the mitral valve via a catheter in a percutaneous procedure.

5. A system as in claim 2 also used as an anchor for an artificial mitral valve.

6. An implant as in claim 1 wherein said adjustment is bi-directional.

7. An implant as in claim 1 comprising a shape memory alloy.

8. An implant as in claim 1 comprising a magnetostrictive alloy.

9. An implant as in claim 1 comprising a Terfenol-D alloy.

10. An implant as in claim 1 comprising a Ni—Mn-GA alloy.

11. An implant as in claim 1 wherein said system comprises a piezoelectric material.

12. An implant as in claim 1 comprising of at least two actuators capable of being selectively activated using different parameter of said external magnetic field.

13. A system as in claim 3 wherein a plurality of actuators are attached to the spine allowing non-invasive gradual adjustments.

14. An implant as in claim 1 wherein said implant comprises permanent magnets.

15. An implant as in claim 1 compatible with MRI imaging.

16. A system as in claim 2 compatible with MRI imaging

17. A system as in claim 2 compatible with MRI imaging

18. An implant as in claim 1 comprising a magnetostrictive element placed between two one-way clutches inside a sealed tube, said element capable of changing the dimension of said implant in response to an externally created magnetic field.

19. An implant as in claim 1 comprising a shape memory alloy element placed between two one-way clutches inside a sealed tube, said element capable of changing the dimension of said implant in response to heating induced in a non-invasive manner.
